(19) United States
(12) Patent Application Publication
(10) Pub. No.: US 2026/0248586 A1
(43) Pub. Date: Aug. 27, 2026

(54) INSERTABLE TOOL IDENTIFICATION FOR FLEXIBLE ELONGATE DEVICES

(71) Applicant: INTUITIVE SURGICAL OPERATIONS, INC., Sunnyvale, CA (US)

(72) Inventors: Shibing Liu, Sunnyvale, CA (US); Samuel B. Schorr, San Jose, CA (US); Sang Gyum Kim, San Jose, CA (US)

(21) Appl. No.: 19/469,320

(22) PCT Filed: Mar. 26, 2024
(86) PCT No.: PCT/US24/21404

§ 371 Date: Sep. 25, 2025

Related U.S. Application Data
(60) Provisional application No. 63/492,334, filed on Mar. 27, 2023.

(51) Int. Cl. A61B 90/90; A61B 90/00

(52) U.S. Cl. CPC A61B 90/90; A61B 90/06; A61B 2090/066

(57) ABSTRACT

Medical systems and methods include a flexible elongate device having an articulable body portion and a lumen extending therethrough. A control system utilizes data from one or more sensors to identify a tool inserted into a lumen of the flexible elongate device.

### FIELD

Disclosed embodiments relate to identification of tools for use with flexible elongate devices.

### BACKGROUND

Minimally invasive medical techniques are intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions, physicians may insert minimally invasive medical instruments (including surgical, diagnostic, therapeutic, and/or biopsy instruments) to reach a target tissue location. One such minimally invasive technique is to use a flexible and/or steerable elongate device, such as a flexible catheter, that can be inserted into anatomic passageways and navigated toward a region of interest within the patient anatomy for tool delivery.

### SUMMARY

The following presents a simplified summary of various examples described herein and is not intended to identify key or critical elements or to delineate the scope of the claims.

In some examples, a medical system is disclosed that includes a flexible elongate device having an articulable body portion and a lumen extending through the flexible elongate device for receiving one or more tools. The medical system further includes a first sensor configured to obtain first data associated with a first property of a tool adapted to be inserted into the lumen of the flexible elongate device or a combination of the tool adapted to be inserted into the lumen of the flexible elongate device and the flexible elongate device, and a second sensor configured to obtain second data associated with a second property of at least the tool adapted to be inserted into the lumen of the flexible elongate device. A control system of the medical system is configured to identify a tool inserted into the lumen of the flexible elongate device based on the first and second data.

In some examples, a medical system is disclosed that includes a flexible elongate device having an articulable body portion and a lumen extending through the flexible elongate device for receiving one or more tools. The medical system further includes a sensor configured to obtain data generated by a positional or control deviation of the flexible elongate device as a result of insertion of a tool into the lumen and a control system configured to identify a tool by comparing the data to known tool profiles.

In some examples, a medical system is disclosed that includes a flexible elongate device having an articulable body portion and a lumen extending through the flexible elongate device for receiving one or more tools. The medical system further includes a first sensor configured to obtain identification data associated with a property of a tool adapted to be inserted into the lumen of the flexible elongate device or a combination of the tool adapted to be inserted into the lumen of the flexible elongate device and the flexible elongate device, and a second sensor configured to obtain insertion data generated by insertion of the tool into the lumen of the flexible elongate device. A control system of the medical system is configured to identify a tool profile from a plurality of known tool profiles matching the identification data to identify the tool inserted into the lumen of the flexible elongate device and determine that the tool is fully inserted into the lumen of the flexible elongate device based on the insertion data.

In some examples, a medical system is disclosed that includes a flexible elongate device having an articulable body portion and a lumen extending through the flexible elongate device for receiving one or more tools. The medical system further includes a first induction sensor configured to obtain first induction data associated with a tool adapted to be inserted into the lumen of the flexible elongate device and a second induction sensor configured to obtain second induction data associated with a tool adapted to be inserted into the lumen of the flexible elongate device. A control system of the medical system is configured to identify a tool inserted into the lumen of the flexible elongate device based on a difference between the first and second inductance data.

In some examples, a medical system is disclosed that includes a flexible elongate device having an articulable body portion and a lumen extending through the flexible elongate device for receiving one or more tools. The medical system further includes an induction sensor configured to obtain induction data associated with a tool adapted to be inserted into the lumen of the flexible elongate device and a control system. The control system is configured to determine noise in the induction data from a source other than a tool inserted into the lumen of the flexible elongate device, adjust the induction data to account for the noise, and identify the tool inserted into the lumen of the flexible elongate device based on the adjusted inductance data.

It is to be understood that both the foregoing general description and the following detailed description are illustrative and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1 is a simplified diagram of a medical system according to some embodiments.

FIG. 2A is a simplified diagram of a medical instrument system according to some embodiments.

FIG. 2B is a simplified diagram of a medical instrument including a medical tool within an elongate device according to some embodiments.

FIG. 3 is a simplified diagram of an instrument manipulator including a catheter assembly according to some embodiments.

FIGS. 4A and 4B are simplified diagrams of side views of a patient coordinate space including a medical instrument mounted on an insertion assembly according to some embodiments.

FIG. 5 is a simplified diagram of a medical system according to some embodiments.

FIG. 6 is a flowchart for a method of operating a medical system according to some embodiments.

FIG. 7 is a flowchart for a method of operating a medical system according to some embodiments.

FIG. 8 is a flowchart for a method of operating a medical system according to some embodiments.

FIG. 9 is a flowchart for a method of operating a medical system according to some embodiments.

FIG. 10 is a flowchart for a method of operating a medical system according to some embodiments.

Embodiments of the present disclosure and their advantages are best understood by referring to the detailed description that follows. It should be appreciated that like reference numerals are used to identify like elements illustrated in one or more of the figures, wherein showings therein are for purposes of illustrating embodiments of the present disclosure and not for purposes of limiting the same.

### DETAILED DESCRIPTION

In the following description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional. In some instances, well known methods, procedures, components, and circuits have not been described in detail so as not to unnecessarily obscure aspects of the embodiments.

This disclosure describes various instruments and portions of instruments in terms of their state in three-dimensional space. As used herein, the term “position” refers to the location of an object or a portion of an object in a three-dimensional space (e.g., three degrees of translational freedom along Cartesian x-, y-, and z-coordinates). As used herein, the term “orientation” refers to the rotational placement of an object or a portion of an object (e.g., one or more degrees of rotational freedom such as, roll, pitch, and yaw). As used herein, the term “pose” refers to the position of an object or a portion of an object in at least one degree of translational freedom and to the orientation of that object or portion of the object in at least one degree of rotational freedom (e.g., up to six total degrees of freedom). As used herein, the term “shape” refers to a set of poses, positions, and/or orientations measured along an object. As used herein, the term “distal” refers to a position that is closer to a procedural site and the term “proximal” refers to a position that is further from the procedural site. Accordingly, the distal portion or distal end of an instrument is closer to a procedural site than a proximal portion or proximal end of the instrument when the instrument is being used as designed to perform a procedure.

The systems and methods herein utilize one or more sensors to identify a tool inserted into a lumen of a flexible elongate device. For example, a medical system may include a flexible elongate device, one or more sensors, and a control system. The flexible elongate device includes an articulable body portion and a lumen extending through the flexible elongate device for receiving one or more tools. The system may include an actuator configured to control articulation of the articulable body portion.

Identifying the type of tool inserted within the flexible elongate device may provide improved control, stability, and accuracy for articulation of the flexible elongate device. Furthermore, compiling data from procedures having known tools may provide cumulative information regarding tool efficacy and other procedure improvements. One procedure improvement may be for an output to the user, such as a screen of the system, to automatically switch based on the identified tool type. For example, if a user inserted a radial endobronichial ultrasound (rEBUS) tool, the view may switch from a vision probe to the rEBUS view.

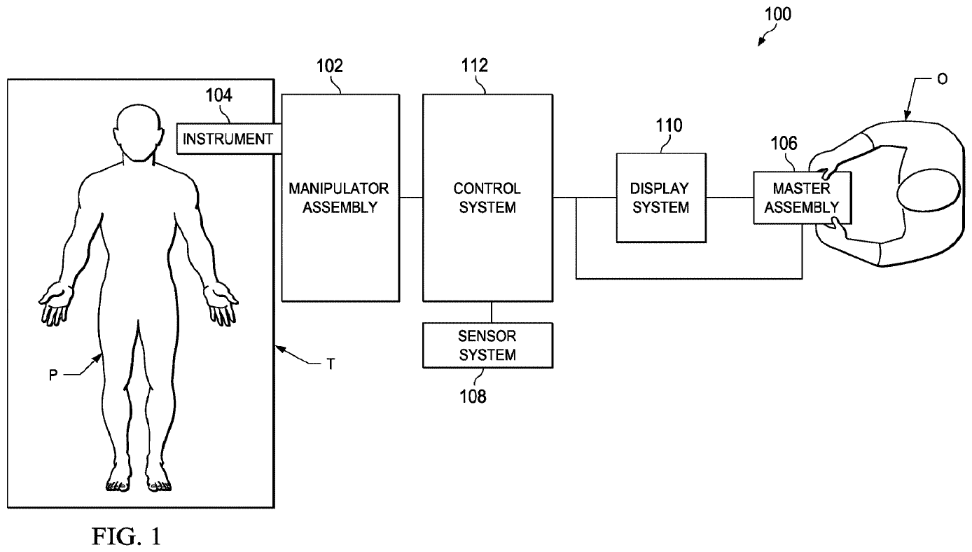

FIG. 1 is a simplified diagram of a medical system 100 according to some embodiments. The medical system 100 may be suitable for use in, for example, surgical, diagnostic (e.g., biopsy), or therapeutic (e.g., ablation, electroporation, etc.) procedures. While some embodiments are provided herein with respect to such procedures, any reference to medical or surgical instruments and medical or surgical methods is non-limiting. The systems, instruments, and methods described herein may be used for animals, human cadavers, animal cadavers, portions of human or animal anatomy, non-surgical diagnosis, as well as for industrial systems, general or special purpose robotic systems, general or special purpose teleoperational systems, or robotic medical systems.

As shown in FIG. 1, medical system 100 may include a manipulator assembly 102 that controls the operation of a medical instrument 104 in performing various procedures on a patient P. Medical instrument 104 may extend into an internal site within the body of patient P via an opening in the body of patient P. The manipulator assembly 102 may be teleoperated, non-teleoperated, or a hybrid teleoperated and non-teleoperated assembly with one or more degrees of freedom of motion that may be motorized and/or one or more degrees of freedom of motion that may be non-motorized (e.g., manually operated). The manipulator assembly 102 may be mounted to and/or positioned near a patient table T. A master assembly 106 allows an operator O (e.g., a surgeon, a clinician, a physician, or other user) to control the manipulator assembly 102. In some examples, the master assembly 106 allows the operator O to view the procedural site or other graphical or informational displays. In some examples, the manipulator assembly 102 may be excluded from the medical system 100 and the instrument 104 may be controlled directly by the operator O. In some examples, the manipulator assembly 102 may be manually controlled by the operator O. Direct operator control may include various handles and operator interfaces for hand-held operation of the instrument 104.

The master assembly 106 may be located at a surgeon's console which is in proximity to (e.g., in the same room as) a patient table T on which patient P is located, such as at the side of the patient table T. In some examples, the master assembly 106 is remote from the patient table T, such as in in a different room or a different building from the patient table T. The master assembly 106 may include one or more control devices for controlling the manipulator assembly 102. The control devices may include any number of a variety of input devices, such as joysticks, trackballs, scroll wheels, directional pads, buttons, data gloves, trigger-guns, hand-operated controllers, voice recognition devices, motion or presence sensors, and/or the like.

The manipulator assembly 102 supports the medical instrument 104 and may include a kinematic structure of links that provide a set-up structure. The links may include one or more non-servo controlled links (e.g., one or more links that may be manually positioned and locked in place) and/or one or more servo controlled links (e.g., one or more links that may be controlled in response to commands, such as from a control system 112). The manipulator assembly 102 may include a plurality of actuators (e.g., motors) that drive inputs on the medical instrument 104 in response to commands, such as from the control system 112. The actuators may include drive systems that move the medical instrument 104 in various ways when coupled to the medical instrument 104. For example, one or more actuators may advance medical instrument 104 into a naturally or surgically created anatomic orifice. Actuators may control articulation of the medical instrument 104, such as by moving the distal end (or any other portion) of medical instrument 104 in multiple degrees of freedom. These degrees of freedom may include three degrees of linear motion (e.g., linear motion along the X, Y, Z Cartesian axes) and in three degrees of rotational motion (e.g., rotation about the X, Y, Z Cartesian axes). One or more actuators may control rotation of the medical instrument about a longitudinal axis. Actuators can also be used to move an articulable end effector of medical instrument 104, such as for grasping tissue in the jaws of a biopsy device and/or the like, or may be used to move or otherwise control tools (e.g., imaging tools, ablation tools, biopsy tools, electroporation tools, etc.) that are inserted within the medical instrument 104.

The medical system 100 may include a sensor system 108 with one or more sub-systems for receiving information about the manipulator assembly 102 and/or the medical instrument 104. Such sub-systems may include a position sensor system (e.g., that uses electromagnetic (EM) sensors or other types of sensors that detect position or location); a shape sensor system for determining the position, orientation, speed, velocity, pose, and/or shape of a distal end and/or of one or more segments along a flexible body of the medical instrument 104; a visualization system (e.g., using a color imaging device, an infrared imaging device, an ultrasound imaging device, an x-ray imaging device, a fluoroscopic imaging device, a computed tomography (CT) imaging device, a magnetic resonance imaging (MRI) imaging device, or some other type of imaging device) for capturing images, such as from the distal end of medical instrument 104 or from some other location; and/or actuator position sensors such as resolvers, encoders, potentiometers, and the like that describe the rotation and/or orientation of the actuators controlling the medical instrument 104.

The medical system 100 may include a display system 110 for displaying an image or representation of the procedural site and the medical instrument 104. Display system 110 and master assembly 106 may be oriented so physician O can control medical instrument 104 and master assembly 106 with the perception of telepresence.

In some embodiments, the medical instrument 104 may include a visualization system, which may include an image capture assembly that records a concurrent or real-time image of a procedural site and provides the image to the operator O through one or more displays of display system 110. The image capture assembly may include various types of imaging devices. The concurrent image may be, for example, a two-dimensional image or a three-dimensional image captured by an endoscope positioned within the anatomical procedural site. In some examples, the visualization system may include endoscopic components that may be integrally or removably coupled to medical instrument 104. Additionally or alternatively, a separate endoscope, attached to a separate manipulator assembly, may be used with medical instrument 104 to image the procedural site. The visualization system may be implemented as hardware, firmware, software or a combination thereof which interact with or are otherwise executed by one or more computer processors, such as of the control system 112.

Display system 110 may also display an image of the procedural site and medical instruments, which may be captured by the visualization system. In some examples, the medical system 100 provides a perception of telepresence to the operator O. For example, images captured by an imaging device at a distal portion of the medical instrument 104 may be presented by the display system 110 to provide the perception of being at the distal portion of the medical instrument 104 to the operator O. The input to the master assembly 106 provided by the operator O may move the distal portion of the medical instrument 104 in a manner that corresponds with the nature of the input (e.g., distal tip turns right when a trackball is rolled to the right) and results in corresponding change to the perspective of the images captured by the imaging device at the distal portion of the medical instrument 104. As such, the perception of telepresence for the operator O is maintained as the medical instrument 104 is moved using the master assembly 106. The operator O can manipulate the medical instrument 104 and hand controls of the master assembly 106 as if viewing the workspace in substantially true presence, simulating the experience of an operator that is physically manipulating the medical instrument 104 from within the patient anatomy.

In some examples, the display system 110 may present virtual images of a procedural site that are created using image data recorded pre-operatively (e.g., prior to the procedure performed by the medical instrument system 200) or intra-operatively (e.g., concurrent with the procedure performed by the medical instrument system 200), such as image data created using computed tomography (CT), magnetic resonance imaging (MRI), positron emission tomography (PET), fluoroscopy, thermography, ultrasound, optical coherence tomography (OCT), thermal imaging, impedance imaging, laser imaging, nanotube X-ray imaging, and/or the like. The virtual images may include two-dimensional, three-dimensional, or higher-dimensional (e.g., including, for example, time based or velocity-based information) images. In some examples, one or more models are created from pre-operative or intra-operative image data sets and the virtual images are generated using the one or more models.

In some examples, for purposes of imaged guided medical procedures, display system 110 may display a virtual image that is generated based on tracking the location of medical instrument 104. For example, the tracked location of the medical instrument 104 may be registered (e.g., dynamically referenced) with the model generated using the pre-operative or intra-operative images, with different portions of the model correspond with different locations of the patient anatomy. As the medical instrument 104 moves through the patient anatomy, the registration is used to determine portions of the model corresponding with the location and/or perspective of the medical instrument 104 and virtual images are generated using the determined portions of the model. This may be done to present the operator O with virtual images of the internal procedural site from viewpoints of medical instrument 104 that correspond with the tracked locations of the medical instrument 104.

The medical system 100 may also include the control system 112, which may include processing circuitry that implements the some or all of the methods or functionality discussed herein. The control system 112 may include at least one memory and at least one processor for controlling the operations of the manipulator assembly 102, the medical instrument 104, the master assembly 106, the sensor system 108, and/or the display system 110. Control system 112 may include instructions (e.g., a non-transitory machine-readable medium storing the instructions) that when executed by the at least one processor, configures the one or more processors to implement some or all of the methods or functionality discussed herein. While the control system 112 is shown as a single block in FIG. 1, the control system 112 may include two or more separate data processing circuits with one portion of the processing being performed at the manipulator assembly 102, another portion of the processing being performed at the master assembly 106, and/or the like. In some examples, the control system 112 may include other types of processing circuitry, such as application-specific integrated circuits (ASICs) and/or field-programmable gate array (FPGAs). The control system 112 may be implemented using hardware, firmware, software, or a combination thereof.

In some examples, the control system 112 may receive feedback from the medical instrument 104, such as force and/or torque feedback. Responsive to the feedback, the control system 112 may transmit signals to the master assembly 106. In some examples, the control system 112 may transmit signals instructing one or more actuators of the manipulator assembly 102 to move the medical instrument 104. In some examples, the control system 112 may transmit informational displays regarding the feedback to the display system 110 for presentation or perform other types of actions based on the feedback.

The control system 112 may include a virtual visualization system to provide navigation assistance to operator O when controlling the medical instrument 104 during an image-guided medical procedure. Virtual navigation using the virtual visualization system may be based upon an acquired pre-operative or intra-operative dataset of anatomic passageways of the patient P. The control system 112 or a separate computing device may convert the recorded images, using programmed instructions alone or in combination with operator inputs, into a model of the patient anatomy. The model may include a segmented two-dimensional or three-dimensional composite representation of a partial or an entire anatomic organ or anatomic region. An image data set may be associated with the composite representation. The virtual visualization system may obtain sensor data from the sensor system 108 that is used to compute an (e.g., approximate) location of the medical instrument 104 with respect to the anatomy of patient P. The sensor system 108 may be used to register and display the medical instrument 104 together with the pre-operatively or intra-operatively recorded images. For example, PCT Publication WO 2016/191298 (published Dec. 1, 2016 and titled “Systems and Methods of Registration for Image Guided Surgery”), which is incorporated by reference herein in its entirety, discloses example systems.

During a virtual navigation procedure, the sensor system 108 may be used to compute the (e.g., approximate) location of the medical instrument 104 with respect to the anatomy of patient P. The location can be used to produce both macro-level (e.g., external) tracking images of the anatomy of patient P and virtual internal images of the anatomy of patient P. The system may include one or more electromagnetic (EM) sensors, fiber optic sensors, and/or other sensors to register and display a medical instrument together with pre-operatively recorded medical images. For example, U.S. Pat. No. 8,900,131 (filed May 13, 2011 and titled “Medical System Providing Dynamic Registration of a Model of an Anatomic Structure for Image-Guided Surgery”), which is incorporated by reference herein in its entirety, discloses example systems.

Medical system 100 may further include operations and support systems (not shown) such as illumination systems, steering control systems, irrigation systems, and/or suction systems. In some embodiments, the medical system 100 may include more than one manipulator assembly and/or more than one master assembly. The exact number of manipulator assemblies may depend on the medical procedure and space constraints within the procedural room, among other factors. Multiple master assemblies may be co-located or they may be positioned in separate locations. Multiple master assemblies may allow more than one operator to control one or more manipulator assemblies in various combinations.

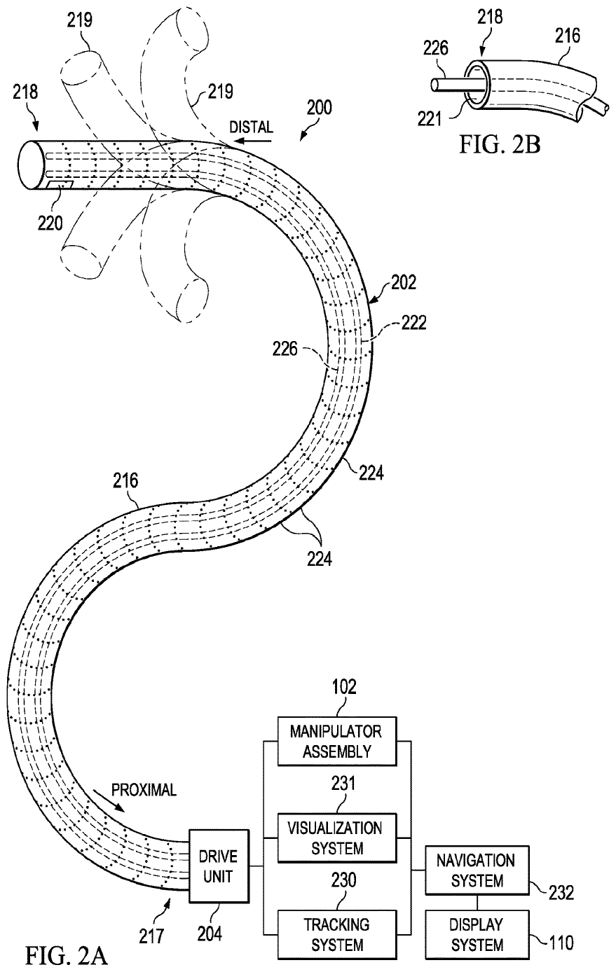

FIG. 2A is a simplified diagram of a medical instrument system 200 according to some embodiments. The medical instrument system 200 includes a flexible elongate device 202 (also referred to as elongate device 202), a drive unit 204, and a medical tool 226 that collectively is an example of a medical instrument 104 of a medical system 100. The medical system 100 may be a teleoperated system, a non-teleoperated system, or a hybrid teleoperated and non-teleoperated system, as explained with reference to FIG. 1. A visualization system 231, tracking system 230, and navigation system 232 are also shown in FIG. 2A and are example components of the control system 112 of the medical system 100. In some examples, the medical instrument system 200 may be used for non-teleoperational exploratory procedures or in procedures involving traditional manually operated medical instruments, such as endoscopy. The medical instrument system 200 may be used to gather (e.g., measure) a set of data points corresponding to locations within anatomic passageways of a patient, such as patient P.

The elongate device 202 is coupled to the drive unit 204. The elongate device 202 includes a channel 221 through which the medical tool 226 may be inserted. The elongate device 202 navigates within patient anatomy to deliver the medical tool 226 to a procedural site. The elongate device 202 includes a flexible body 216 having a proximal end 217 and a distal end 218. In some examples, the flexible body 216 may have an approximately 3 mm outer diameter. Other flexible body outer diameters may be larger or smaller.

Medical instrument system 200 may include the tracking system 230 for determining the position, orientation, speed, velocity, pose, and/or shape of the flexible body 216 at the distal end 218 and/or of one or more segments 224 along flexible body 216, as will be described in further detail below. The tracking system 230 may include one or more sensors and/or imaging devices. The flexible body 216, such as the length between the distal end 218 and the proximal end 217, may include multiple segments 224. The tracking system 230 may be implemented using hardware, firmware, software, or a combination thereof. In some examples, the tracking system 230 is part of control system 112 shown in FIG. 1.

Tracking system 230 may track the distal end 218 and/or one or more of the segments 224 of the flexible body 216 using a shape sensor 222. The shape sensor 222 may include an optical fiber aligned with the flexible body 216 (e.g., provided within an interior channel of the flexibly body 216 or mounted externally along the flexible body 216). In some examples, the optical fiber may have a diameter of approximately 200 μm. In other examples, the diameter may be larger or smaller. The optical fiber of the shape sensor 222 may form a fiber optic bend sensor for determining the shape of flexible body 216. Optical fibers including Fiber Bragg Gratings (FBGs) may be used to provide strain measurements in structures in one or more dimensions. Various systems and methods for monitoring the shape and relative position of an optical fiber in three dimensions, which may be applicable in some embodiments, are described in U.S. Patent Application Publication No. 2006/0013523 (filed Jul. 13, 2005 and titled “Fiber optic position and shape sensing device and method relating thereto”); U.S. Pat. No. 7,772,541 (filed on Mar. 12, 2008 and titled “Fiber Optic Position and/or Shape Sensing Based on Rayleigh Scatter”); and U.S. Pat. No. 8,773,650 (filed on Sept. 2, 2010 and titled “Optical Position and/or Shape Sensing”), which are all incorporated by reference herein in their entireties. Sensors in some embodiments may employ other suitable strain sensing techniques, such as Rayleigh scattering, Raman scattering, Brillouin scattering, and Fluorescence scattering.

In some examples, the shape of the flexible body 216 may be determined using other techniques. For example, a history of the position and/or pose of the distal end 218 of the flexible body 216 can be used to reconstruct the shape of flexible body 216 over an interval of time (e.g., as the flexible body 216 is advanced or retracted within a patient anatomy). In some examples, the tracking system 230 may alternatively and/or additionally track the distal end 218 of the flexible body 216 using a position sensor system 220. Position sensor system 220 may be a component of an EM sensor system with the position sensor system 220 including one or more position sensors. Although the position sensor system 220 is shown as being near the distal end 218 of the flexible body 216 to track the distal end 218, the number and location of the position sensors of the position sensor system 220 may vary to track different regions along the flexible body 216. In one example, the position sensors include conductive coils that may be subjected to an externally generated electromagnetic field. Each coil of position sensor system 220 may produce an induced electrical signal having characteristics that depend on the position and orientation of the coil relative to the externally generated electromagnetic field. The position sensor system 220 may measure one or more position coordinates and/or one or more orientation angles associated with one or more portions of flexible body 216. In some examples, the position sensor system 220 may be configured and positioned to measure six degrees of freedom, e.g., three position coordinates X, Y, Z and three orientation angles indicating pitch, yaw, and roll of a base point. In some examples, the position sensor system 220 may be configured and positioned to measure five degrees of freedom, e.g., three position coordinates X, Y, Z and two orientation angles indicating pitch and yaw of a base point. Further description of a position sensor system, which may be applicable in some embodiments, is provided in U.S. Pat. No. 6,380,732 (filed Aug. 11, 1999 and titled “Six-Degree of Freedom Tracking System Having a Passive Transponder on the Object Being Tracked”), which is incorporated by reference herein in its entirety.

In some embodiments, the tracking system 230 may alternately and/or additionally rely on a collection of pose, position, and/or orientation data stored for a point of an elongate device 202 and/or medical tool 226 captured during one or more cycles of alternating motion, such as breathing. This stored data may be used to develop shape information about the flexible body 216. In some examples, a series of position sensors (not shown), such as EM sensors like the sensors in position sensor 220 or some other type of position sensors may be positioned along the flexible body 216 and used for shape sensing. In some examples, a history of data from one or more of these position sensors taken during a procedure may be used to represent the shape of elongate device 202, particularly if an anatomic passageway is generally static.

FIG. 2B is a simplified diagram of the medical tool 226 within the elongate device 202 according to some embodiments. The flexible body 216 of the elongate device 202 may include the channel 221 sized and shaped to receive the medical tool 226. In some embodiments, the medical tool 226 may be used for procedures such as diagnostics, imaging, surgery, biopsy, ablation, illumination, irrigation, suction, electroporation, etc. Medical tool 226 can be deployed through channel 221 of flexible body 216 and operated at a procedural site within the anatomy. Medical tool 226 may be, for example, an image capture probe, a biopsy tool (e.g., a needle, grasper, brush, etc.), an ablation tool (e.g., a laser ablation tool, radio frequency (RF) ablation tool, cryoablation tool, thermal ablation tool, heated liquid ablation tool, etc.), an electroporation tool, and/or another surgical, diagnostic, or therapeutic tool. In some examples, the medical tool 226 may include an end effector having a single working member such as a scalpel, a blunt blade, an optical fiber, an electrode, and/or the like. Other end types of end effectors may include, for example, forceps, graspers, scissors, staplers, clip appliers, and/or the like. Other end effectors may further include electrically activated end effectors such as electrosurgical electrodes, transducers, sensors, and/or the like.

The medical tool 226 may be a biopsy tool used to remove sample tissue or a sampling of cells from a target anatomic location. In some examples, the biopsy tool is a flexible needle. The biopsy tool may further include a sheath that can surround the flexible needle to protect the needle and interior surface of the channel 221 when the biopsy tool is within the channel 221. The medical tool 226 may be an image capture probe that includes a distal portion with a stereoscopic or monoscopic camera that may be placed at or near the distal end 218 of flexible body 216 for capturing images (e.g., still or video images). The captured images may be processed by the visualization system 231 for display and/or provided to the tracking system 230 to support tracking of the distal end 218 of the flexible body 216 and/or one or more of the segments 224 of the flexible body 216. The image capture probe may include a cable for transmitting the captured image data that is coupled to an imaging device at the distal portion of the image capture probe. In some examples, the image capture probe may include a fiber-optic bundle, such as a fiberscope, that couples to a more proximal imaging device of the visualization system 231. The image capture probe may be single-spectral or multi-spectral, for example, capturing image data in one or more of the visible, near-infrared, infrared, and/or ultraviolet spectrums. The image capture probe may also include one or more light emitters that provide illumination to facilitate image capture. In some examples, the image capture probe may use ultrasound, x-ray, fluoroscopy, CT, MRI, or other types of imaging technology.

In some examples, the image capture probe is inserted within the flexible body 216 of the elongate device 202 to facilitate visual navigation of the elongate device 202 to a procedural site and then is replaced within the flexible body 216 with another type of medical tool 226 that performs the procedure. In some examples, the image capture probe may be within the flexible body 216 of the elongate device 202 along with another type of medical tool 226 to facilitate simultaneous image capture and tissue intervention, such as within the same channel 221 or in separate channels. A medical tool 226 may be advanced from the opening of the channel 221 to perform the procedure (or some other functionality) and then retracted back into the channel 221 when the procedure is complete. The medical tool 226 may be removed from the proximal end 217 of the flexible body 216 or from another optional instrument port (not shown) along flexible body 216.

In some examples, the elongate device 202 may include integrated imaging capability rather than utilize a removable image capture probe. For example, the imaging device (or fiber-optic bundle) and the light emitters may be located at the distal end 218 of the elongate device 202. The flexible body 216 may include one or more dedicated channels that carry the cable(s) and/or optical fiber(s) between the distal end 218 and the visualization system 231. Here, the medical instrument system 200 can perform simultaneous imaging and tool operations.

In some examples, the medical tool 226 is capable of controllable articulation. The medical tool 226 may house cables (which may also be referred to as pull wires), linkages, or other actuation controls (not shown) that extend between its proximal and distal ends to controllably bend the distal end of medical tool 226, such as discussed herein for the flexible elongate device 202. The medical tool 226 may be coupled to a drive unit 204 and the manipulator assembly 102. In these examples, the elongate device 202 may be excluded from the medical instrument system 200 or may be a flexible device that does not have controllable articulation. Steerable instruments or tools, applicable in some embodiments, are further described in detail in U.S. Pat. No. 7,316,681 (filed on Oct. 4, 2005 and titled “Articulated Surgical Instrument for Performing Minimally Invasive Surgery with Enhanced Dexterity and Sensitivity”) and U.S. Pat. No. 9,259,274 (filed Sept. 30, 2008 and titled “Passive Preload and Capstan Drive for Surgical Instruments”), which are incorporated by reference herein in their entireties.

The flexible body 216 of the elongate device 202 may also or alternatively house cables, linkages, or other steering controls (not shown) that extend between the drive unit 204 and the distal end 218 to controllably bend the distal end 218 as shown, for example, by broken dashed line depictions 219 of the distal end 218 in FIG. 2A. In some examples, at least four cables are used to provide independent up-down steering to control a pitch of the distal end 218 and left-right steering to control a yaw of the distal end 281. In these examples, the flexible elongate device 202 may be a steerable catheter. Examples of steerable catheters, applicable in some embodiments, are described in detail in PCT Publication WO 2019/018736 (published Jan. 24, 2019 and titled “Flexible Elongate Device Systems and Methods”), which is incorporated by reference herein in its entirety.

In embodiments where the elongate device 202 and/or medical tool 226 are actuated by a telcoperational assembly (e.g., the manipulator assembly 102), the drive unit 204 may include drive inputs that removably couple to and receive power from drive elements, such as actuators, of the teleoperational assembly. In some examples, the elongate device 202 and/or medical tool 226 may include gripping features, manual actuators, or other components for manually controlling the motion of the elongate device 202 and/or medical tool 226. The elongate device 202 may be steerable or, alternatively, the elongate device 202 may be non-steerable with no integrated mechanism for operator control of the bending of distal end 218. In some examples, one or more channels 221 (which may also be referred to as lumens), through which medical tools 226 can be deployed and used at a target anatomical location, may be defined by the interior walls of the flexible body 216 of the elongate device 202.

In some examples, the medical instrument system 200 (e.g., the elongate device 202 or medical tool 226) may include a flexible bronchial instrument, such as a bronchoscope or bronchial catheter, for use in examination, diagnosis, biopsy, and/or treatment of a lung. The medical instrument system 200 may also be suited for navigation and treatment of other tissues, via natural or surgically created connected passageways, in any of a variety of anatomic systems, including the colon, the intestines, the kidneys and kidney calices, the brain, the heart, the circulatory system including vasculature, and/or the like.

The information from the tracking system 230 may be sent to the navigation system 232, where the information may be combined with information from the visualization system 231 and/or pre-operatively obtained models to provide the physician, clinician, surgeon, or other operator with real-time position information. In some examples, the real-time position information may be displayed on the display system 110 for use in the control of the medical instrument system 200. In some examples, the navigation system 232 may utilize the position information as feedback for positioning medical instrument system 200. Various systems for using fiber optic sensors to register and display a surgical instrument with surgical images, applicable in some embodiments, are provided in U.S. Pat. No. 8,900,131 (filed May 13, 2011 and titled “Medical System Providing Dynamic Registration of a Model of an Anatomic Structure for Image-Guided Surgery”), which is incorporated by reference herein in its entirety.

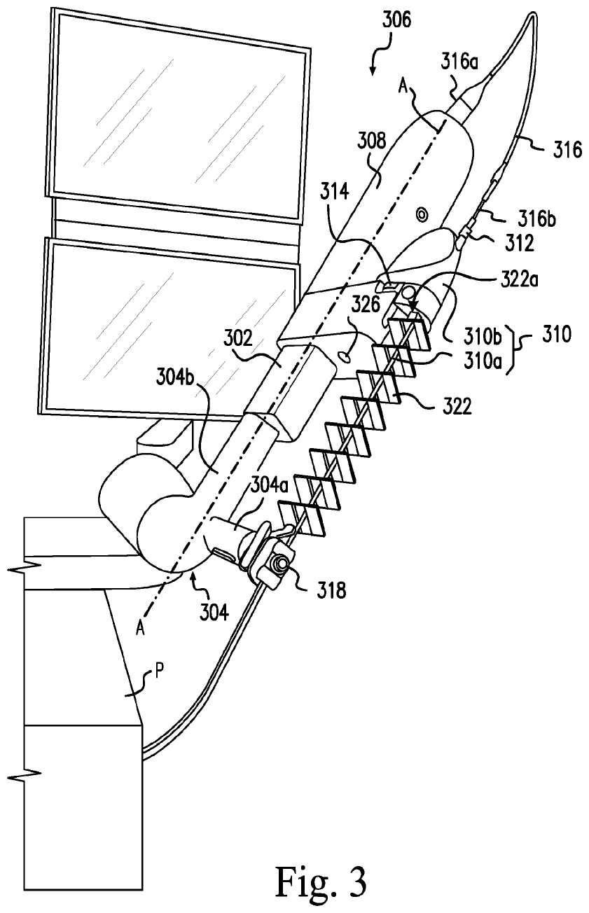

FIG. 3 shows an example of an instrument manipulator 306, which may be substantially similar to the instrument manipulator 206. The instrument manipulator 306 may include a base 304, an insertion stage 302, and an instrument carriage 308 to which a catheter assembly 310 is coupled. In one or more embodiments, the instrument manipulator 306 provides for insertion and retraction of the catheter assembly 310, with respect to the patient anatomy, by moving the instrument carriage 308 and insertion stage 302 in a telescoping manner relative to the base 304. The instrument manipulator 306, thus, provides an insertion degree of freedom for the insertion and retraction of the flexible catheter 310a. In a medical scenario, the insertion may advance the flexible catheter 310a into the patient anatomy, whereas the retraction may withdraw the flexible catheter 310a from the patient anatomy.

The base 304 includes a shaft portion 304a and a main portion 304b. As described in detail below, the shaft portion 304a removably couples to a device connector or swivel connector 318 which receives the flexible catheter 310a. The insertion stage 302 is coupled to the main portion 304b of the base 304 and translates along the main portion 304b. The instrument carriage 308 is coupled to and translates along the insertion stage 302. The catheter assembly 310 may include a flexible catheter 310a and a control assembly 310b. The instrument carriage 308 couples to the control assembly 310b at an instrument interface 314 of the instrument carriage 308. The instrument manipulator 306 also couples to a probe assembly 316 which includes a probe 316b and a probe connector 316a. The probe assembly 316 may insert into a working lumen of the flexible catheter 310a through the connector 312 on the control assembly 310b and may run through the flexible catheter 310a. The probe 316b may include, for example, a viewing scope assembly that provides images of a surgical site. The instrument carriage 308 may include electronic and optical components providing probe 316b with endoscopic capabilities. In some embodiments, the probe assembly 316 may be detached from the instrument manipulator 306 and flexible catheter control assembly 310b, and removed from the catheter assembly 310. Alternative instruments such as biopsy needles, ablation tools, and other flexible instruments may be coupled to the instrument manipulator 306 and/or the catheter assembly 310, through the flexible catheter 310a working lumen.

Continuing with FIG. 3, the device connector or swivel connector 318 may include a manipulator interface which may be removably coupled to the base 304. In some embodiments, the flexible catheter 310a runs through a catheter guide 322, which is a selectively collapsible and extendable device that supports the length of the flexible catheter 310a during movement of the instrument carriage 308. The flexible catheter 310a without guidance may buckle in regions with no lateral support, e.g., in the space between the instrument interface 314 and the device connector 318. To avoid the buckling, the catheter guide 322 may be an anti-buckling guide by providing lateral support to the flexible catheter 310a. Various systems and methods related to catheter guides are described in PCT/US2017/041160 (filed Jul. 7, 2017) (disclosing “Guide Apparatus for Delivery of an Elongate Device and Methods of Use”), which is incorporated by reference herein in its entirety.

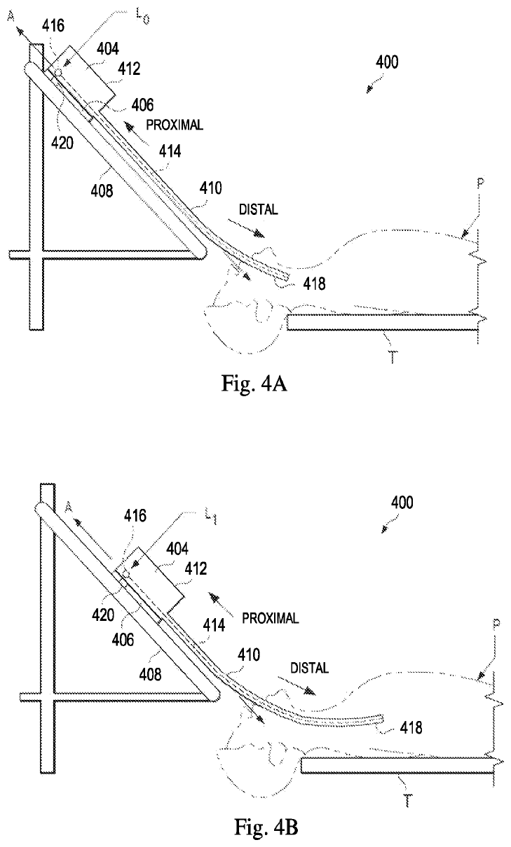

FIGS. 4A and 4B are simplified diagrams of side views of a patient coordinate space including a medical instrument mounted on an insertion assembly according to some embodiments. As shown in FIGS. 4A and 4B, a surgical environment 400 may include a patient P positioned on the patient table T. Patient P may be stationary within the surgical environment 400 in the sense that gross patient movement is limited by sedation, restraint, and/or other means. Cyclic anatomic motion, including respiration and cardiac motion, of patient P may continue. Within surgical environment 400, a medical instrument 404 is used to perform a medical procedure which may include, for example, surgery, biopsy, ablation, illumination, irrigation, suction, or electroporation. The medical instrument 404 may also be used to perform other types of procedures, such as a registration procedure to associate the position, orientation, and/or pose data captured by the sensor system 108 to a desired (e.g., anatomical or system) reference frame. The medical instrument 404 may be, for example, the medical instrument 104. In some examples, the medical instrument 404 may include an elongate device 410 (e.g., a catheter) coupled to an instrument body 412. Elongate device 410 includes one or more channels sized and shaped to receive a medical tool.

Elongate device 410 may also include one or more sensors (e.g., components of the sensor system 108). In some examples, a shape sensor 414 may be fixed at a proximal point 416 on the instrument body 412. The proximal point 416 of the shape sensor 414 may be movable with the instrument body 412, and the location of the proximal point 416 with respect to a desired reference frame may be known (e.g., via a tracking sensor or other tracking device). The shape sensor 414 may measure a shape from the proximal point 416 to another point, such as a distal end 418 of the elongate device 410. The shape sensor 414 may be aligned with the elongate device 410 (e.g., provided within an interior channel or mounted externally). In some examples, the shape sensor 414 may optical fibers used to generate shape information for the elongate device 410.

In some examples, position sensors (e.g., EM sensors) may be incorporated into the medical instrument 404. A series of position sensors may be positioned along the flexible elongate device 410 and used for shape sensing. Position sensors may be used alternatively to the shape sensor 414 or with the shape sensor 414, such as to improve the accuracy of shape sensing or to verify shape information.

Elongate device 410 may house cables, linkages, or other steering controls that extend between the instrument body 412 and the distal end 418 to controllably bend the distal end 418. In some examples, at least four cables are used to provide independent up-down steering to control a pitch of distal end 418 and left-right steering to control a yaw of distal end 418. The instrument body 412 may include drive inputs that removably couple to and receive power from drive elements, such as actuators, of a manipulator assembly.

The instrument body 412 may be coupled to an instrument carriage 406. The instrument carriage 406 may be mounted to an insertion stage 408 that is fixed within the surgical environment 400. Alternatively, the insertion stage 408 may be movable but have a known location (e.g., via a tracking sensor or other tracking device) within surgical environment 400. Instrument carriage 406 may be a component of a manipulator assembly (e.g., manipulator assembly 102) that couples to the medical instrument 404 to control insertion motion (e.g., motion along an insertion axis A) and/or motion of the distal end 418 of the elongate device 410 in multiple directions, such as yaw, pitch, and/or roll. The instrument carriage 406 or insertion stage 408 may include actuators, such as servomotors, that control motion of instrument carriage 406 along the insertion stage 408.

A sensor device 420, which may be a component of the sensor system 108, may provide information about the position of the instrument body 412 as it moves relative to the insertion stage 408 along the insertion axis A. The sensor device 420 may include one or more resolvers, encoders, potentiometers, and/or other sensors that measure the rotation and/or orientation of the actuators controlling the motion of the instrument carriage 406, thus indicating the motion of the instrument body 412. In some embodiments, the insertion stage 408 has a linear track as shown in FIGS. 4A and 4B. In some embodiments, the insertion stage 408 may have curved track or have a combination of curved and linear track sections.

FIG. 4A shows the instrument body 412 and the instrument carriage 406 in a retracted position along the insertion stage 408. In this retracted position, the proximal point 416 is at a position L0 on the insertion axis A. The location of the proximal point 416 may be set to a zero value and/or other reference value to provide a base reference (e.g., corresponding to the origin of a desired reference frame) to describe the position of the instrument carriage 406 along the insertion stage 408. In the retracted position, the distal end 418 of the elongate device 410 may be positioned just inside an entry orifice of patient P. Also in the retracted position, the data captured by the sensor device 420 may be set to a zero value and/or other reference value (e.g., I=0). In FIG. 4B, the instrument body 412 and the instrument carriage 406 have advanced along the linear track of insertion stage 408, and the distal end 418 of the elongate device 410 has advanced into patient P. In this advanced position, the proximal point 416 is at a position L1 on the insertion axis A. In some examples, the rotation and/or orientation of the actuators measured by the sensor device 420 indicating movement of the instrument carriage 406 along the insertion stage 408 and/or one or more position sensors associated with instrument carriage 406 and/or the insertion stage 408 may be used to determine the position L1 of the proximal point 416 relative to the position LO. In some examples, the position L1 may further be used as an indicator of the distance or insertion depth to which the distal end 418 of the elongate device 410 is inserted into the passageway(s) of the anatomy of patient P.

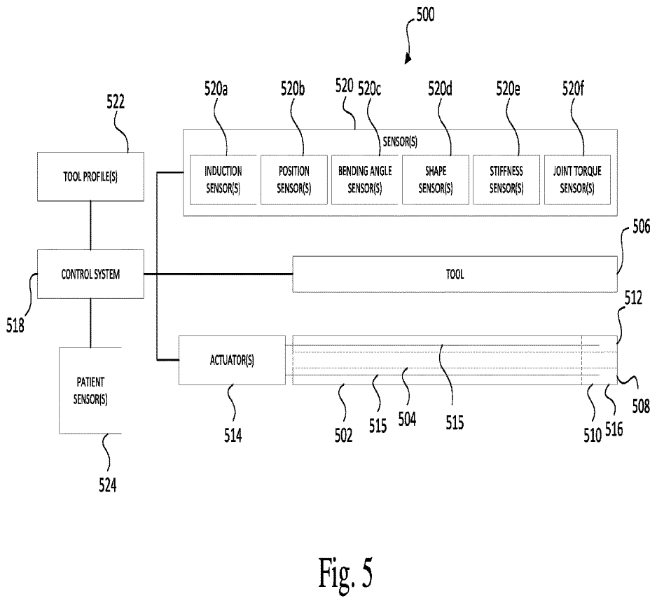

FIG. 5 is a simplified diagram of a medical instrument system 500 including a flexible elongate device 502. According to some embodiments consistent with FIGS. 1-4, the medical instrument system 500 may correspond to the medical instrument system 200 and/or the flexible elongate device 502 may correspond to the elongate device 202.

The flexible elongate device 502 can include a flexible body and a main lumen 504 that extends through the flexible body. The main lumen 504 may provide a delivery channel for a medical tool 506. The medical tool 506 can be any suitable tool, including, for example, a vision probe, a biopsy tool (e.g., a needle, brush, cryoprobe, or forceps), an ablation tool, an electroporation tool, an ultrasound device (e.g., endobronchial ultrasound (EBUS) probe), a chemical delivery tool, and/or other biopsy or treatment tools, to be inserted through the flexible body of the flexible elongate device 502.

As shown in FIG. 5, the flexible body of the flexible elongate device 502 can include an articulable body portion 508, which may be in a distal section 510 of the body including a distal tip 512 thereof. In the illustrated embodiment, the system 500 includes one or more actuators 514 that control articulation of the articulable body portion 508 via manipulation of one or more control elements 515, such as pull wires, tendons, push rods, and/or the like, connected to a control structure 516 (e.g., control ring) of the articulable body portion 508. Operation of the actuator 514 causes the respective control element to pull back to cause the articulable body portion 508 to bend in the direction of the control element or release allowing the articulable body portion 508 to return to a straighter configuration.

The system 500 includes a control system 518 that is operably coupled to the actuator(s) 514 to thereby control manipulation of positioning of the articulable body portion 508 of the flexible elongate device 502. The system 500 further includes one or more sensors 520 coupled or disposed adjacent to the flexible elongate device 502 to help the control system 518 identify a tool 506 insertable into the lumen 504 of the flexible elongate device 502. In some embodiments, the systems and methods provided herein may utilize the sensor(s) 520 to obtain data associated with the tool 506 insertable into the lumen 504 of the flexible elongate device 502 and/or data associated with the tool 506 in combination with the flexible elongate device 502.

The different structural configurations of different types of tools 506 may be utilized for identification purposes. In one example, the different structural configurations of the tools 506 may result in different induction profiles along a length of the tools 506. As such, the induction profiles may be used to identify a particular tool (e.g., tool type) or narrow the applicable tools. In other examples, different types of tools 506 may have different physical properties, resulting in a different overall properties for the combined flexible elongate device 502 and tool 506, including, for example, stiffness, inertia, friction, and so forth. These differences have different impacts on the flexible elongate device-tool system 500, such that a particular change (e.g., amount of deflection of the articulable body portion 508, a torque change required to hold the articulable body portion 508 at a desired bending angle, a stiffness change in the combined flexible elongate device 502 and tool 506 as compared to the flexible elongate device 502 alone, shape changes of the flexible elongate device 502 while the tool 506 is being inserted into the flexible elongate device 502, etc.) can be correlated by the control system 518 to a particular tool for identification purposes. The combined stiffness of the flexible elongate device 502 and tool 506 affects the manipulation of the articulable body portion 508 by requiring greater amounts of force to achieve desired bending angles. The combined inertia of the flexible elongate device 502 and tool 506 results in more delayed responses to manipulation of the articulable body portion 508, greater amounts of force to begin manipulation movements, and greater amounts of force to stop movement of the articulable body portion 508 when it has reached a desired bending angle. As the tool 506 is inserted into the flexible elongate device 502, friction builds up between the tool 506 and an interior surface of the lumen 504 of the flexible elongate device 502. A pathway of the flexible elongate device 502 within a patient may also affect the amount of combined friction. For example, more circuitous routes within a patient creates additional acute bends, each of which adds to the overall friction between the device 502 and tool 506. Due to this accumulation, additional force is required to overcome the friction and achieve a desired bending angle for the articulable body portion 508. The systems and methods provided herein may also utilize one or more of these aspects within a tool profile to identify a type of tool insertable within the flexible elongate device 502. Alternatively, or additionally, the systems and methods provided herein may use one or more identified properties to adjust control of the flexible elongate device 502 without identifying a particular tool. For example, control of the flexible elongate device 502 can be controlled according to a determined combined stiffness, combined inertia, combined friction, etc.

The data provided from the sensors 520 may help identify a particular tool 506 in several ways. In one example, the sensors 520 may include one or more inductive sensors 520a to measure inductance of the tool 506 along a length of the tool 506, e.g., at periodic or predetermined locations, continuously, etc., while the tool 506 is inserted within the flexible elongate device 502 or before the tool 506 is inserted within the flexible elongate device 502. The length of the tool 506 may be a proximal portion, a distal portion, and/or an intermediate portion of the tool 506.

Additionally, when the tool 506 is inserted within the flexible elongate device 502 a position of at least a portion of the flexible elongate device 502 may change. Position changes can include a position change of the (e.g., distal) articulable body portion 508 of the flexible elongate device 502, a bending angle change of the articulable body portion 508, or an overall shape change of the flexible elongate device 502 in response to the tool insertion. Furthermore, insertion of the tool 506 may change the combined stiffness of the flexible elongate device 502 and tool 506 and/or change a force required to hold the articulable body portion 508 at a desired bending angle. Accordingly, data obtained or measured by the sensors 520 may also or alternatively include: position change data, bending angle change data, shape change data, stiffness change data, and/or torque change data.

In additional or alternative examples, the sensors 520 may include one or more of the following: one or more sensors 520b to measure a position of a portion of the flexible elongate device 502, such as the articulable body portion 508, in space, one or more sensors 520c to measure a bending angle of the articulable body portion 508 of the flexible elongate device 502, one or more sensors 520d to measure the shape of the flexible elongate device 502 along some or all of a length of the flexible elongate device 502, one or more sensors 520e to measure a stiffness of the flexible elongate device 502 with the tool 506 inserted therein and, in some embodiments, without the tool 506 inserted therein, and/or one or more sensors 520f to measure a joint torque to hold the articulable body portion 508 with the tool 506 extending through the lumen 504 at a predetermined bending angle.

Any suitable sensors 520, including, for example, inductive sensors, fiber shape sensors, force sensors, magnetic sensors, etc., may be used to measure the above data. In some embodiments, the one or more sensors 520b to measure the position of the portion of the flexible elongate device 502 in space may include fiber shape sensors and/or magnetic sensors, the one or more sensors 520c to measure the bending angle of the articulable body portion 508 of the flexible elongate device 502 can include fiber shape sensors and/or magnetic sensors, the one or more sensors 520d to measure the shape of the flexible elongate device 502 along its length may include fiber shape sensors and/or magnetic sensors, the one or more sensors 520e to measure the stiffness of the flexible elongate device 502 and tool 506 may include force sensors operably coupled to measure the torque of the actuator(s) 514 of the system 500/motor encoders for the actuator(s) 514 of the system 500, a fiber shape sensor, and a model of the flexible elongate device 502, and the one or more sensors 520f to measure a joint torque to hold the articulable body portion 508 with the tool 506 extending through the lumen 504 at a predetermined bending angle may include force sensors operably coupled to measure the torque of the actuator(s) 514 of the system 500. It will be understood that the sensors 520a-520f are shown in FIG. 5 separately for ease of description and with reference to different measurement items; however, one or more sensors can be used to measure two, three, or more items. For example, the sensor(s) can be used to measure position, bending angle, and shape.

The sensors 520 can be coupled (e.g., mounted directly or indirectly) to the flexible elongate device 502, to other components of the system 500 (e.g., the actuator(s) 514), or be disposed in locations remote from the flexible elongate device 502, tool 506, or other components of the system 500 to obtain the particular data for the flexible elongate device 502 and/or tool 506.

In some examples, the induction sensors 520a can be coupled to the flexible elongate device 502, such as embedded within the flexible elongate device 502, or coupled to another portion of the system 500 through which the tool 506 is inserted, such as the backend of the flexible elongate device, catheter anti-buckling guide, swivel connector, endotracheal tube, etc.

In some examples, the position sensors 520b, the bending angle sensors 520c, and/or the shape sensors 520d can be coupled to the flexible elongate device 502, such as embedded within the flexible elongate device 502. The sensors 520b, 520c, 520d can be coupled to any desired portion of the flexible elongate device 502, such as within the articulable body portion 508, an intermediate portion, and/or a proximal portion. One or more of the sensors 520b, 520c, 520d can be a shape sensor (e.g., fiber shape sensor), one or more position sensors (e.g., electromagnetic sensors), and/or an encoder for a motor of the actuator(s) 514 along with a model for the flexible elongate device 502 (e.g., a shape sensor generated model) that can provide a bending angle/position for the articulable body portion 508 using the encoder data associated with operation of the actuator(s) 514 as input.

In some examples, the stiffness sensors 520e can include a shape sensor (e.g., fiber shape sensor) to provide ground truth bending angle data of the articulable body portion 508 along with an encoder for a motor of the actuator(s) 514/torque data for the actuator(s) 514 and a model for the flexible elongate device 502 that can convert the encoder data/torque to joint torque of the system 500 to maintain the bending angle of the articulable body portion 508. The control system 518 can estimate a stiffness by dividing the joint torque by the bending angle.

In some examples, the joint torque sensors 520f can be motor current sensors coupled to a manipulator (e.g., manipulator assembly 102) to measure current supplied to the motors of the actuator(s) 514. Advantageously, motor current sensors can provide data on the effects of inertia, friction, and pullwire torque in the system, with and without the tool 506. In other or additional examples, the joint torque sensors 520f can be a torque sensor coupled to the pullwire side of the manipulator at the proximal end of the flexible elongate device 502 to measure torque applied to the pullwires or other control elements of the flexible elongate device 502.

In some embodiments, the system 500 may include or access one or more tool profiles that contain information about particular tools. Data in the tool profiles may be collected prior to a procedure and/or compiled from one or more previous procedures. Each tool profile may include data that corresponds to the data obtained or measured by the sensors 520 of the system 500, such as induction data or position data, either directly or with further processing as described herein. Pursuant to this, each tool profile may include: an induction profile of the flexible elongate device 502, a position change profile of the flexible elongate device 502, a bending angle deflection profile of the articulable body portion 508, a shape deviation profile of the flexible elongate device 502 associated with insertion of the tool 506 into the flexible elongate device 502, a stiffness profile for the articulable body portion 508 of the flexible elongate device 502 with the tool 506 inserted therein, and/or a torque profile associated with holding the articulable body portion 508 with the tool 506 extending therethrough at one or more bending angles.

In some examples, the induction profile includes inductance data including a layout of metallic objects along a predetermined length of the tool 506. The layout can include spacing of the metallic objects relative to one another, as well as sizes of the metallic objects. The layout may correspond to one or more portions of the tool's 506 length, such a forward portion, a rear portion, or an intermediate portion, or may correspond to the tool's 506 entire length (e.g., insertable length). As the tool 506 passes the induction sensor(s) 520a (e.g., as it is being inserted into the flexible elongate device 502), the measured induction will change with changes in the construction of the tool 506 and the induction profile would correspond to what is expected to be measured by the sensor(s) 520a. In some instances, the tool 506 can be identified based on steady state amplitude and inductance change patterns (e.g., positive/negative step changes). Pursuant to this, the inductance data and/or induction profile can include the following information to identify tool type: a final inductance state, a state indicator vector, first and second inductance states to determine whether the inductance goes up or down, and a total number of active states in the state indicator vector. The tool identification algorithm may use a transient response and, as such, in such an example, the tool detection can follow a particular order (e.g., the above order of information).

In some examples, the position change profile can include position data in the form of position change data associated with how the position of one or more portions of the flexible elongate device 502, such as the articulable body portion 508, one or more intermediate portions, and/or one or more proximal portions, changes in space upon insertion of the tool 506 within the flexible elongate device 502, or retraction of the tool 506 within the flexible elongate device 502. The position change data can also correspond to a particular bending angle that the articulable body portion 508 was in prior to deflection due to insertion of the tool 506. The bending angle can include a range of bending angles. In some embodiments, the range of bending angles includes any number of angles from 1 degree to 180 degrees. As set forth, the position change profile can include position change of portions of the flexible elongate device 502 other than the articulable body portion 508, including proximal and/or intermediate portions of the flexible elongate device 502. As the flexible elongate device 502 is inserted into a patient, the device 502 may be guided through a number of twists and turns, causing the device 502 to have corresponding bends. The position change of one or more these bends as the tool 506 is inserted therethrough can be correlated to a particular tool or tools.

In some examples, the bending angle deflection profile can include position data in the form of deflection data for the particular tool 506 with regard to an amount of bending angle deflection that the tool 506 causes the articulable body portion 508 to move from one or more beginning bending angles. The one or more beginning bending angles can include a range of beginning bending angles with corresponding deflection data. In some embodiments, the range of beginning bending angles includes any number of angles from 1 degree to 180 degrees. In additional or alternative examples, the bending angle deflection profile can include bending angle deflections of portions of the flexible elongate device 502 other than the articulable body portion 508, including proximal and/or intermediate portions of the flexible elongate device 502. As the flexible elongate device 502 is inserted into a patient, the device 502 may be guided through a number of twists and turns, causing the device 502 to have corresponding bends. The deflection of the angles of one or more these bends as the tool 506 is inserted therethrough can be correlated to a particular tool or tools.

In some examples, the shape deviation profile can include position data in the form of shape change data that includes shape changes that occur along a length of the flexible elongate device 502 as the tool 506 is inserted into the flexible elongate device 502. For example, as the tool 506 is guided to bends in the flexible elongate device 502 extending within anatomical pathways within a patient, the stiffness of the tool 506 will cause the flexible elongate device 502 to deflect or cause the sidewall of the flexible elongate device 502 to expand outwardly slightly. The changes to the shape of the flexible elongate device 502 that result from insertion of the tool 506 into the flexible elongate device 502 at one or more curves/bending angles can be compiled into the shape deviation profile. The one or more curves/bending angles can include a range of curves/bending angles with corresponding deflection data. In some embodiments, the range of bending angles includes any number of angles from 1 degree to 180 degrees.

In some examples, the stiffness profile can include stiffness data for the particular tool 506 based on joint torque and bending angle data provided by a shape sensor. The stiffness profile may include the stiffness data, one or more joint torque and bending angle combinations for the particular tool, or combinations thereof. The one or more bending angles can include a range of bending angles. In some embodiments, the range of bending angles includes any number of angles from 1 degree to 180 degrees.

In some examples, the torque profile can include torque data for the particular tool 506 with regard to an amount of torque needed from the actuator(s) 514 to hold the articulable body portion 508 with the tool 506 extending therethrough at one or more bending angles. The one or more bending angles can include a range of bending angles. In some embodiments, the range of bending angles includes any number of angles from 1 degree to 180 degrees.

In other examples, the data can be correlated directly to particular tools (e.g., tool types) without reference to an overall tool profile. The data can include position data that may include one or more of the position change data, the bending angle deflection data, and/or the shape deviation data. The data can also, or alternatively, include one or more of the inductance data, the stiffness data and/or the torque data.

In one implementation, data from the sensors 520, such as two, three, or more, is utilized by the control system 518 to identify a tool based on a plurality of properties. For example, a first sensor 520 may obtain data associated with a first property of the tool 506 adapted to be inserted into the lumen 504 of the flexible elongate device 502 or the combined tool and flexible elongate device and a second sensor 520 may obtain data associated with a second property of the tool 506 or the combined tool and flexible elongate device. The first and second properties are different to provide different identification features of the tool 506. The control system 518 may then identify the tool 506 inserted into the lumen 504 of the flexible elongate device 502 based on the first and second data.

In one example, the first sensor 520 may be an induction sensor 520a and the second sensor 520 may be a sensor 520 different from the induction sensor 520a, such as one or more position sensors 520b, bending angle sensors 520c, shape sensors 520d, stiffness sensors 520e, or joint torque sensors 520f. With this configuration, the first property may be induction profiles for one or more tools and the second property may be profiles corresponding to data from the other sensing method. In further examples, additional sensors 520 may provide data associated with a third property, a fourth property, etc.

The first and second properties may allow the control system 518 to identify the tool in any suitable way. In one example, the control system 518 may determine a tool profile 522 having first and second properties matching or closely correlating to the data from the first and second sensors 520 from a plurality of tool profiles 522 (e.g., a tool profile for each type of tool insertable into the flexible elongate device 502.) In another example, data from the first sensor 520 associated with the first property may allow the control system 518 to narrow down possible tools and data from the second sensor 520 may allow the control system 518 to identify a particular tool from the previously-narrowed possible tools. Stated another way, the control system 518 may identify one or more tool profiles 522 matching the data from the first sensor 520 and identify a single tool profile 522 of the one or more tool profiles 522 matching the data from the second sensor 520 to identify the tool 506 inserted into the lumen 504 of the flexible elongate device 502. The system 500 may be further expanded, if desired, to include a third or more sensors 520 to provide additional data, and narrowing of the possible tools, for the tool determination. The sensors 520 of this example may include any combination of sensors 520a-520f discussed above, with the associated data and profiles.

It will be understood that the data may correspond to the tool 506 individually or a combination of the tool 506 and the flexible elongate device 502. For example, the first data may be associated with a first property of only the tool 506 adapted to be inserted into the lumen 504 of the flexible elongate device 502. Alternatively, the first data may be associated with a first property of the tool 506 adapted to be inserted into the lumen 504 of the flexible elongate device 502 in combination with the flexible elongate device 502. Similarly, the second data may be associated with a second property of only the tool 506 adapted to be inserted into the lumen 504 of the flexible elongate device 502. Alternatively, the second data may be associated with a second property of the tool 506 adapted to be inserted into the lumen 504 of the flexible elongate device 502 in combination with the flexible elongate device 502.

In another implementation, the sensor 520 may provide data generated by deviation of the flexible elongate device 502 as a result of insertion of the tool 506 into the lumen 504 thereof. Stated another way, the sensor 520 can be configured to obtain data generated by a positional (e.g., bending angle, position, or shape changes for the flexible elongate device 502) or control (e.g., torque changes to control articulation of the articulable body portion 508) deviation (e.g., an amount of deviation) of the flexible elongate device 502 as a result of insertion of the tool 506 into the lumen 504. The stiffness, inertia, friction, and other inherent properties of the tool 506 impact the position and control of the flexible elongate device 502 when the tool 506 is inserted therein. The control system 518 can use this data to identify the tool 506 by comparing the data to known tool profiles 522.

In this example, the positional or control deviation data measured or obtained by the sensor 520 as a result of the tool 506 being inserted into the flexible elongate device 502 may include bending angle change data, position change data, torque change data, stiffness change data, and/or shape change data, as described above. The data can reflect changes in the articulable body portion 508 or other flexible body portions of the flexible elongate device 502. As such, the sensors 520 of this example may be any one or combination of the sensors 520b-520f discussed above (e.g., a fiber shape sensor, a force sensor, or a magnetic sensor).

Each tool profile of this example may include: a position change profile of the flexible elongate device 502 including the articulable body portion 508 thereof, a bending angle deflection profile of the flexible elongate device 502 including the articulable body portion 508 thereof, a shape deviation profile of the flexible elongate device 502 associated with insertion of the tool 506 into the flexible elongate device 502, a stiffness profile for the articulable body portion 508 of the flexible elongate device 502 with the tool 506 inserted therein, and/or a torque profile associated with holding the articulable body portion 508 with the tool 506 extending therethrough at one or more bending angles.

In some configurations, locations for coupling an induction sensor 520a to the system 500 may include structure that produces noise in the inductance data observed by the induction sensor 520a. For example, the induction sensor 520a may be coupled to a control assembly (e.g., control assembly 310b), an instrument carriage (e.g., instrument carriage 406) or other non-teleoperational manipulators or other structures used for receiving a tool. As discussed above, the system 500 may include one or more pull wires 515 that are tensioned via operation of the one or actuators 514 to control articulation of the articulable body portion 508. The system 500 may further include coil pipes or similar support structure for the pull wires 515 that may cause data observed by an induction sensor to have noise. For example, it has been found that catheter motion (e.g., due to catheter bending angle change or human breathing during a procedure) can have an impact on inductance change. With this configuration, pull wire tension change can result in an inductance/capacitance change in the coil pipe and/or the flexible elongate device.

Pursuant this and in another example, the first sensor 520 is a first induction sensor 520a and the second sensor 520 is a second induction sensor 520a. To identify the tool 506, the control system 518 can determine a difference between first inductance data from the first induction sensor 520a and second inductance data from the second induction sensor 520a. By determining the difference between the inductance data, the control system 518 can cancel out or reduce noise from a source observed by both the first and second induction sensors 520a (e.g., a same source).

The first and second induction sensors 520a can be spaced from one another a distance along a tool insertion path of the system 500. In one implementation, the sensors 520a can be disposed close enough to one another to receive the same noise, but far enough apart that there is a delay in the data from the sensors 520a. For example, the sensors 520a can be coupled to any suitable components of any of the systems described herein, such as an anti-buckling guide, a control assembly configured to support and position the flexible elongate device 502, an instrument carriage, or combinations thereof (e.g., anti-buckling guide 322, instrument carriage 308, control assembly 310b). In one example, the first and second sensors 520a can be located in a spaced relation relative to one another at a proximal or top end 322a of the anti-buckling guide 322.

Being able to determine the difference between the inductance data of spaced sensors helps the system adjust for a patient's breathing frequency or other types of motion that may occur during a procedure. Further, the configuration may help the system adjust for the effects of tension, including within the flexible elongate device 502 and associated components, such as coil pipes or other support structure, pull wires, etc.

In another approach, if the induction data noise is known or can be estimated, a single induction sensor 520a may be utilized rather than using the difference between two inductance sensors 520a. The control system 518 can be configured to determine the noise in the induction data from a source other than the tool 506 and adjust the signal to account for the noise. The noise can result from the effects of motion and/or tension, for example. After the induction data is adjusted to account from the noise, the control system 518 can identify the tool 506 based on the adjusted induction data, as described herein.

For example, if a patient's breathing frequency is known, the control system 518 can filter out the noise based on the frequency. In one implementation, one or more sensors 524 (FIG. 5) may monitor patient data (e.g., a patient's breathing/respiration rate and timing), and send data to the control system 518 accordingly. By aligning the respiration rate of the patient over the induction data received from the induction sensor 520a, the control system 518 can adjust the induction data in coordination with the respiration rate to provide filtered data that can be utilized to identify the tool 506.

In another or alterative example, the control system 518 may dynamically adjust for motion in the flexible elongate device 502 by resetting the baseline inductance in the system. An amount of bending motion imparted to the articulable body portion 508 can be correlated to an inductance change and the control system 518 can update the baseline inductance for the system to thereby interpret the induction data from the sensor 520a without the noise associated with motion of the flexible elongate device 502. In some embodiments, the control system 518 can store a range of bending motions/angles with corresponding inductance change values to dynamically update the baseline inductance of the system during a procedure.

In some embodiments, the systems and methods herein may utilize one or more sensors 520 to identify when the tool 506 has been fully inserted into the lumen 504 of the flexible elongate device 502 in addition to utilizing one or more sensors 520 to identify the tool 506. As such, in these embodiments, the system 500 may include one or more first sensors 520 configured to obtain identification data associated with a property of the tool 506 adapted to be inserted into the lumen 504 of the flexible elongate device 502 or a property of the combined tool and flexible elongate device. The system 500 further includes one or more second sensors 520 configured to obtain insertion data generated by insertion of the tool 506 into the lumen 504 thereof.

The property of the tool 506 from the identification data may allow the control system 518 to identify the tool 506 (e.g., tool type) in any suitable way. In one example, the control system 518 may identify the tool based on the identification data. In another example, the control system 518 may determine a tool profile 522 having a property matching or closely correlating to the identification data from the first sensor 520 from a plurality of tool profiles 522 (e.g., a tool profile for each type of tool insertable into the flexible elongate device 502.) The sensor 520 for the identification data of this example may be any of the sensors 520-520f discussed above. The system 500 may also be expanded, if desired, to include a second, third, or more sensors 520 to provide additional identification data, and narrowing of the possible tools, for the tool determination according to any of the concepts described herein.

In examples utilizing tool profiles, as set forth in more detail above, each tool profile may include: an induction profile along a length of the flexible elongate device 502, a position change profile of the articulable body portion 508, a bending angle deflection profile of the articulable body portion 508, a shape deviation profile of the flexible elongate device 502 associated with insertion of the tool 506 into the flexible elongate device 502, a stiffness profile for the articulable body portion 508 of the flexible elongate device 502 with the tool 506 inserted therein, and/or a torque profile associated with holding the articulable body portion 508 with the tool 506 extending therethrough at one or more bending angles.

In addition to identifying the tool 506 being inserted into the flexible elongate device 502, the control system 518 of the medical system 500 is further configured to determine that the tool 506 is fully inserted into the lumen 504 of the flexible elongate device 502 based on the insertion data from the second sensor 520.

In some examples, the insertion data may be positional or control deviation data generated by deviation of the flexible elongate device 502 as a result of insertion of the tool 506 into the lumen 504 thereof. In this example, similar to the above embodiment, the positional or control deviation data measured or obtained by the sensor 520 may include bending angle change data, position change data, torque change data, stiffness change data, and/or shape change data, as described above. As such, the second sensor 520 may be a fiber shape sensor, a force sensor, or a magnetic sensor. In these examples and with the articulable body portion 508 being the distal section 510 of the flexible elongate device 502, the control system 518 may determine that the tool 506 is fully inserted within the flexible elongate device 502 on receiving the positional or control deviation data, as the positional or control deviation data indicates that the tool 506 is disposed through and influencing the position or control of the articulable body portion 508.

In additional or alternative examples, the insertion data may be inductance data and the second sensor 520 may be an inductive sensor 520a to measure inductance of the tool 506 along a proximal portion of the tool 506 that corresponds to the tool 506 being fully inserted within the flexible elongate device 502. In this example, the control system 518 may compare the inductance data with one or more induction profiles for known tools. As discussed above, the induction profiles may include a layout of metallic objects along a predetermined length (e.g., proximal portion) of the tool 506.

In one example, the first sensor to identify the tool 506 may be an induction sensor 520a to measure an induction of the tool 506 and the second sensor may be a shape sensor to identify bending angle and/or position change of the articulable body portion 508 of the flexible elongate device 502. In another example, the first sensor to identify the tool 506 may be an induction sensor 520a to measure an induction of the tool 506 and the second sensor may be a torque sensor. When the tool 506 is inserted through the articulable body portion 508, the control system 518 may adjust a torque of the actuator(s) 514 to bring the bending angle from a deviated position due to the tool 506 back to a controlled position. As such, the torque change data may be analyzed to determine if the tool 506 is fully seated within the flexible elongate device 502.

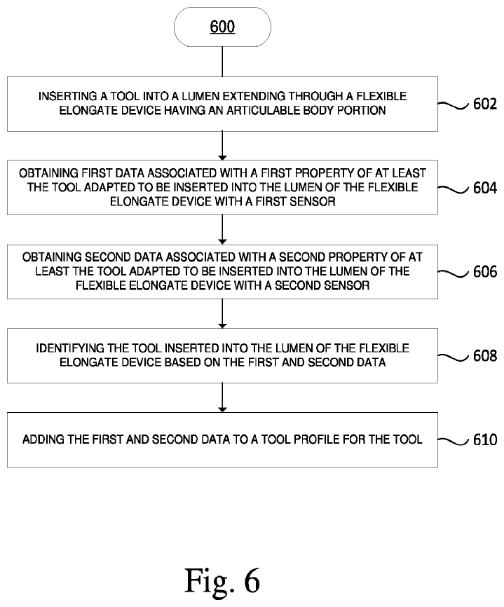

FIG. 6 illustrates a method 600 for operation of a medical system including a flexible elongate device (e.g., the medical system 500 and flexible elongate device 502) according to some embodiments. The method 600 is illustrated as a set of operations or processes 602 through 610. Not all of the illustrated processes may be performed in all embodiments of the method 600. Additionally, one or more processes that are not expressly illustrated in FIG. 6 may be included before, after, in between, or as part of the processes 602 through 610. Processes may also be performed in different orders. In some embodiments, one or more of the processes 602 through 610 may be implemented, at least in part, in the form of executable code stored on non-transitory, tangible, machine-readable media that when run by one or more processors (e.g., the processors of a controller) may cause the one or more processors to perform one or more of the processes. In one or more embodiments, the processes 602 through 610 may be performed by a controller.

In process 602, a tool (e.g., tool 506) is inserted into a lumen extending through a flexible elongate device having an articulable body portion (e.g., flexible elongate device 502 having lumen 504 and articulable body portion 508). In process 604, a first sensor (e.g., sensor 520) obtains first data associated with a first property of at least the tool adapted to be inserted into the lumen of the flexible elongate device and, in process 606, a second sensor (e.g., sensor 520) obtains second data associated with a second property of at least the tool adapted to be inserted into the lumen of the flexible elongate device. In process 608, a control system (e.g., control system 518) identifies the tool inserted into the lumen of the flexible elongate device based on the first and second data. In process 610, the control system adds the first and second data to a tool profile (e.g., tool profile 522) for the tool to build the tool profile.

In some examples, process 608 can include selecting a tool profile from a plurality of tool profiles based on the first and second data to identify the tool; identifying a plurality of matching tool profiles based on the first data and identifying a tool profile for the tool from the plurality of matching tool profiles based on the second data; and/or identifying an unapproved tool inserted into the lumen of the flexible elongate device based on the first and second data.

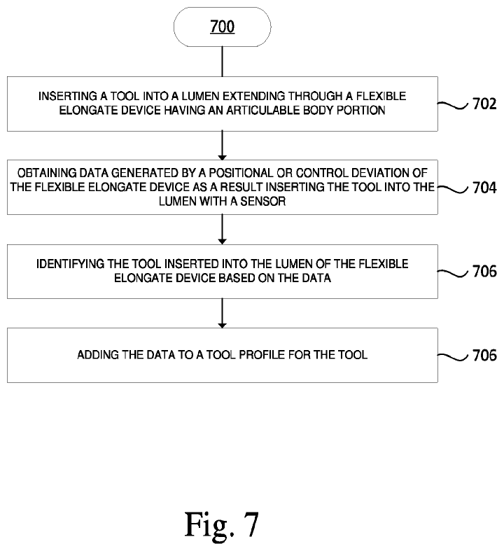

FIG. 7 illustrates a method 700 for operation of a medical system including a flexible elongate device (e.g., the medical system 500 and flexible elongate device 502) according to some embodiments. The method 700 is illustrated as a set of operations or processes 702 through 708. Not all of the illustrated processes may be performed in all embodiments of the method 700. Additionally, one or more processes that are not expressly illustrated in FIG. 7 may be included before, after, in between, or as part of the processes 702 through 708. Processes may also be performed in different orders. In some embodiments, one or more of the processes 702 through 708 may be implemented, at least in part, in the form of executable code stored on non-transitory, tangible, machine-readable media that when run by one or more processors (e.g., the processors of a controller) may cause the one or more processors to perform one or more of the processes. In one or more embodiments, the processes 702 through 708 may be performed by a controller.

In process 702, a tool (e.g., tool 506) is inserted into a lumen extending through a flexible elongate device having an articulable body portion (e.g., flexible elongate device 502 having lumen 504 and articulable body portion 508). In process 704, a sensor (e.g., sensor 520) obtains data generated by a positional or control deviation of the flexible elongate device as a result of process 702. In process 706, a control system (e.g., control system 518) identifies the tool inserted into the lumen of the flexible elongate device based on the data. For example, identification of the tool can be achieved by comparing the data to known tool profiles (e.g., tool profiles 522). In process 708, the control system adds the data to a tool profile for the tool 506 to build the tool profile. In one example, process 706 can include identifying an unapproved tool inserted into the lumen of the flexible elongate device based on the first and second data.

The control system may also adjust control of the flexible elongate device 502 based on the data. The adjustment may fully or partially account for properties of the combined flexible elongate device 502 and tool 506. For example, the tool 506 may add stiffness, inertia, and/or friction to the system 500, which results in different control inputs to position the articulable body portion 508 at desired positions and bending angles. The data itself can be utilized as input to adjust control or, in examples identifying one or more tool profiles, the tool profile can be utilized as input to adjust control. The systems and methods described herein may also implement tool behavior modifications based on recognition of a tool.

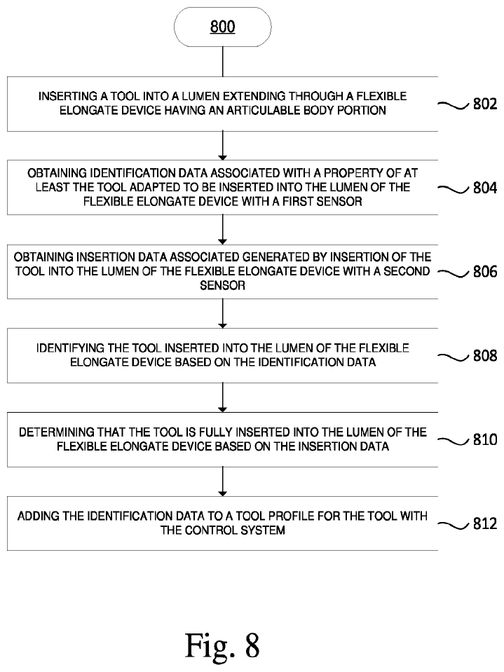

FIG. 8 illustrates a method 800 for operation of a medical system including a flexible elongate device (e.g., the medical system 500 and flexible elongate device 502) according to some embodiments. The method 800 is illustrated as a set of operations or processes 802 through 812. Not all of the illustrated processes may be performed in all embodiments of the method 800. Additionally, one or more processes that are not expressly illustrated in FIG. 8 may be included before, after, in between, or as part of the processes 802 through 812. Processes may also be performed in different orders. In some embodiments, one or more of the processes 802 through 812 may be implemented, at least in part, in the form of executable code stored on non-transitory, tangible, machine-readable media that when run by one or more processors (e.g., the processors of a controller) may cause the one or more processors to perform one or more of the processes. In one or more embodiments, the processes 802 through 812 may be performed by a controller.

In process 802, a tool (e.g., tool 506) is inserted into a lumen extending through a flexible elongate device having an articulable body portion (e.g., flexible elongate device 502 having lumen 504 and articulable body portion 508). In process 804, a first sensor (e.g., sensor 520) obtains identification data associated with a property of at least the tool adapted to be inserted into the lumen of the flexible elongate device. In process 806, a second sensor (e.g., sensor 520) obtains insertion data associated generated by insertion of the tool into the lumen of the flexible elongate device. In process 808, a control system (e.g., control system 518) identifies the tool inserted into the lumen of the flexible elongate device based on the identification data. For example, identification of the tool can be achieved by identifying a tool profile from a plurality of known tool profiles matching the identification data. In process 810, the control system determines that the tool is fully inserted into the lumen of the flexible elongate device based on the insertion data. In process 812, the control system adds the identification data to a tool profile (e.g., tool profile 522) for the tool 506 to build the tool profile. In some examples, process 808 can include identifying an unapproved tool inserted into the lumen of the flexible elongate device based on the first and second data.

It will be understood that the sensor(s) for any of the above methods 600, 700, 800 may be sensors 520a-520f as described herein.

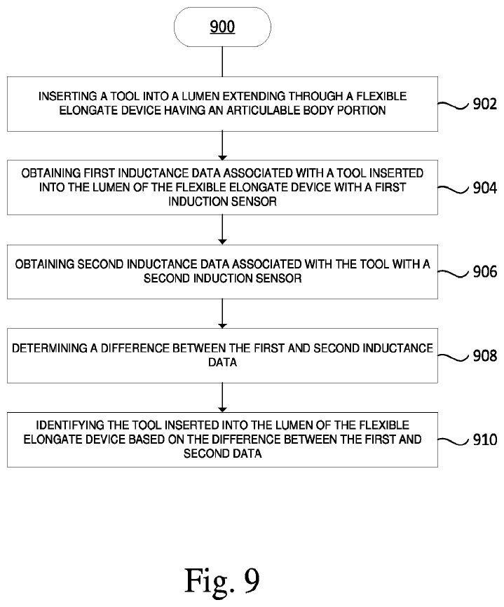

FIG. 9 illustrates a method 900 for operation of a medical system including a flexible elongate device (e.g., the medical system 500 and flexible elongate device 502) according to some embodiments. The method 900 is illustrated as a set of operations or processes 902 through 910. Not all of the illustrated processes may be performed in all embodiments of the method 900. Additionally, one or more processes that are not expressly illustrated in FIG. 9 may be included before, after, in between, or as part of the processes 902 through 910. Processes may also be performed in different orders. In some embodiments, one or more of the processes 902 through 910 may be implemented, at least in part, in the form of executable code stored on non-transitory, tangible, machine-readable media that when run by one or more processors (e.g., the processors of a controller) may cause the one or more processors to perform one or more of the processes. In one or more embodiments, the processes 902 through 910 may be performed by a controller.

In process 902, a tool (e.g., tool 506) is inserted into a lumen extending through a flexible elongate device having an articulable body portion (e.g., flexible elongate device 502 having lumen 504 and articulable body portion 508). In process 904, a first induction sensor (e.g., sensor 520a) obtains first induction data associated with the tool and, in process 906, a second induction sensor (e.g., sensor 520a) obtains second induction data associated with the tool. In process 908, a control system (e.g., control system 518) determines a difference between the first and second inductance data and, in process 910, identifies the tool inserted into the lumen of the flexible elongate device based on the difference between the first and second inductance data.

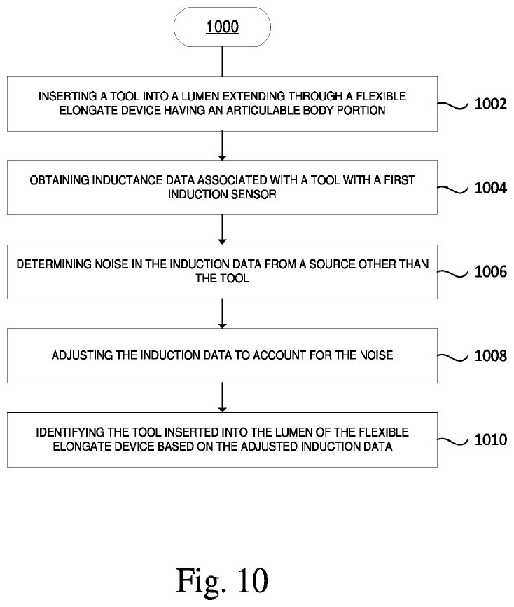

FIG. 10 illustrates a method 1000 for operation of a medical system including a flexible elongate device (e.g., the medical system 500 and flexible elongate device 502) according to some embodiments. The method 1000 is illustrated as a set of operations or processes 1002 through 1010. Not all of the illustrated processes may be performed in all embodiments of the method 1000. Additionally, one or more processes that are not expressly illustrated in FIG. 10 may be included before, after, in between, or as part of the processes 1002 through 1010. Processes may also be performed in different orders. In some embodiments, one or more of the processes 1002 through 1010 may be implemented, at least in part, in the form of executable code stored on non-transitory, tangible, machine-readable media that when run by one or more processors (e.g., the processors of a controller) may cause the one or more processors to perform one or more of the processes. In one or more embodiments, the processes 1002 through 1010 may be performed by a controller.

In process 1002, a tool (e.g., tool 506) is inserted into a lumen extending through a flexible elongate device having an articulable body portion (e.g., flexible elongate device 502 having lumen 504 and articulable body portion 508). In process 1004, an induction sensor (e.g., sensor 520a) obtains induction data associated with the tool. In process 1006, a control system (e.g., control system 518) determines noise in the induction data from a source other than the tool (e.g., breathing or other patient movement and/or tension) and, in process 1008, the control system adjusts the induction data to account for the noise. In process 1010, the control system identifies the tool inserted into the lumen of the flexible elongate device based on the adjusted induction data.

In any of the above systems and/or methods, the control system can also output an identification of the tool 506 (e.g., tool type), a status of the tool 506, and/or data collected to a user of the system 500. For example, the control system can cause the identification and/or status of the tool 506 to be displayed on a local or remote display. In an additional or alternative example, the control system can send a message to a user over any desired communication network (e.g., WiFi, Bluetooth, near filed communication, radio, etc.). The control system can also be receptive to a confirmation or modification input from the user for tool identification. For example, the display can include a corresponding user input (e.g., touch screen, mouse, keyboard, etc.) allowing the user to confirm the tool identification or enter a different tool identification. In another example, the user can respond to the message with a reply message to the control system confirming the tool identification or providing a different tool identification. The data collected and/or the identification of the tool may be recorded as a summary to users.

In some examples, the control system can change a procedure workflow based on an identification of the tool. The procedure workflow may include a plurality of stages including navigation to a target location within a patient. Upon identification of the tool, the control system can change the procedure workflow to a next stage corresponding to use of the tool. For example, if a biopsy tool is inserted, the control system can transition from the navigation state to a biopsy state of the system 500. Similar transitions can be made for other tools, such as an ablation state for an ablation probe, an inspection state for a vision probe, an ultrasound state for an ultrasound device, a delivery state for a chemical delivery tool, or a treatment state for an electroporation tool.

Any of the systems and methods described herein may also be utilized to recognize or detect counterfeit, competitor, or otherwise unauthorized devices or tools (such as a device or tool manufactured by a competitor or an unauthorized manufacturer). For example, the unauthorized devices may have corresponding tool profiles with data corresponding to any combination of the sensors 520a-520f and the control system 518 may detect the unauthorized device by comparing data from the sensor(s) 520 of the system 500 to the unauthorized device tool profile.

Any of the systems and methods described herein may also build tool profiles for the various tools 506 of the system 500 over time. For example, after the control system 518 identifies a particular tool, data collected from the procedure from any of the sensors 520 of the system 500 may be added to the tool profile for the tool 506 to allow the control system 518 to more accurately identify the tool 506 during future procedures.

One or more components of the embodiments discussed in this disclosure, such as control system 112, 518, may be implemented in software for execution on one or more processors of a computer system. The software may include code that when executed by the one or more processors, configures the one or more processors to perform various functionalities as discussed herein. The code may be stored in a non-transitory computer readable storage medium (e.g., a memory, magnetic storage, optical storage, solid-state storage, etc.). The computer readable storage medium may be part of a computer readable storage device, such as an electronic circuit, a semiconductor device, a semiconductor memory device, a read only memory (ROM), a flash memory, an erasable programmable read only memory (EPROM); a floppy diskette, a CD-ROM, an optical disk, a hard disk, or other storage device. The code may be downloaded via computer networks such as the Internet, Intranet, etc. for storage on the computer readable storage medium. The code may be executed by any of a wide variety of centralized or distributed data processing architectures. The programmed instructions of the code may be implemented as a number of separate programs or subroutines, or they may be integrated into a number of other aspects of the systems described herein. The components of the computing systems discussed herein may be connected using wired and/or wireless connections. In some examples, the wireless connections may use wireless communication protocols such as Bluetooth, near-field communication (NFC), Infrared Data Association (IrDA), home radio frequency (HomeRF), IEEE 802.11, Digital Enhanced Cordless Telecommunications (DECT), and wireless medical telemetry service (WMTS).

Various general-purpose computer systems may be used to perform one or more processes, methods, or functionalities described herein. Additionally or alternatively, various specialized computer systems may be used to perform one or more processes, methods, or functionalities described herein. In addition, a variety of programming languages may be used to implement one or more of the processes, methods, or functionalities described herein.

While certain embodiments and examples have been described above and shown in the accompanying drawings, it is to be understood that such embodiments and examples are merely illustrative and are not limited to the specific constructions and arrangements shown and described, since various other alternatives, modifications, and equivalents will be appreciated by those with ordinary skill in the art.

1.-7. (canceled)

8. A medical system comprising:
a flexible elongate device having an articulable body portion and a lumen extending through the flexible elongate device for receiving one or more tools;
a first sensor configured to obtain first data associated with a first property of at least a tool adapted to be inserted into the lumen of the flexible elongate device;
a second sensor configured to obtain second data associated with a second property of at least the tool adapted to be inserted into the lumen of the flexible elongate device; and
a control system configured to identify a tool inserted into the lumen of the flexible elongate device based on the first and second data.

9. The medical system of claim 8, wherein the first sensor comprises a shape or position sensor configured to obtain position data and the second sensor comprises an induction sensor configured to obtain inductance data; and the control system is configured to identify the tool based on the position data and the inductance data.

10. The medical system of claim 8, wherein the first sensor comprises a first induction sensor configured to obtain first inductance data and the second sensor comprises a second induction sensor configured to obtain second inductance data; and the control system is configured to identify the tool based on a difference between the first and second inductance data.

11. The medical system of claim 8, wherein the control system configured to identify the tool inserted into the lumen of the flexible elongate device comprises the control system configured to select a tool profile from a plurality of tool profiles based on the first and second data to identify the tool.

12. The medical system of claim 8, wherein the control system configured to identify the tool inserted into the lumen of the flexible elongate device comprises the control system configured to:
identify a plurality of matching tool profiles based on the first data; and
identify a tool profile for the tool from the plurality of matching tool profiles based on the second data.

13. The medical system of claim 11, wherein each tool profile comprises one or more of: an induction profile along at least a portion of a length of the flexible elongate device, a position change profile of the articulable body portion of the flexible elongate device, a bending angle deflection profile of the articulable body portion of the flexible elongate device, a shape deviation profile of the flexible elongate device associated with insertion of the tool into the flexible elongate device, a stiffness profile for the articulable body portion of the flexible elongate device with the tool inserted therein, or a torque profile associated with holding the articulable body portion of the flexible elongate device with the tool extending therethrough at one or more bending angles.

14. The medical system of claim 11, wherein the control system is further configured to add the first and second data to the tool profile.

15. The medical system of claim 8, wherein the first data is associated with a first property of only the tool adapted to be inserted into the lumen of the flexible elongate device.

16. The medical system of claim 8, wherein the first data is associated with a first property of the tool adapted to be inserted into the lumen of the flexible elongate device in combination with the flexible elongate device.

17. The medical system of claim 8, wherein the second data is associated with a second property of the tool adapted to be inserted into the lumen of the flexible elongate device in combination with the flexible elongate device.

18.-19. (canceled)

20. The medical system of claim 8, further comprising an actuator configured to control manipulation of the articulable body portion of the flexible elongate device, wherein at least one of the first sensor or the second sensor comprises a force sensor configured to measure torque of the actuator.

21. The medical system of claim 8, further comprising a third sensor configured to obtain third data associated with a third property of at least the tool adapted to be inserted into the lumen of the flexible elongate device.

22. The medical system of claim 8, wherein the articulable body portion includes a distal tip of the flexible elongate device.

23. (canceled)

24. The medical system of claim 8, wherein the control system configured to identify the tool inserted into the lumen of the flexible elongate device comprises the control system configured to identify an unapproved tool inserted into the lumen of the flexible elongate device based on the first and second data.

25.-51. (canceled)

52. The medical system of claim 8, further comprising an actuator configured to control articulation of the articulable body portion of the flexible elongate device; and wherein the first sensor comprises an encoder or a torque sensor for the actuator configured to obtain torque data and the second sensor comprises a shape sensor configured to obtain bending angle data of the articulable body portion; and the control system is configured to: estimate a stiffness of at least the tool based on the torque data and the bending angle data; and identify the tool based on the estimated stiffness.

53. A method for identifying a tool inserted into a lumen of a flexible elongate device, the method comprising:
obtaining first data associated with a first property of at least a tool inserted into the lumen of the flexible elongate device with a first sensor;
obtaining second data associated with a second property of at least the tool inserted into the lumen of the flexible elongate device with a second sensor; and
identifying the tool based on the first and second data.

54. The method of claim 53, wherein:
obtaining the first data with the first sensor comprises obtaining position data with a shape or position sensor; and
obtaining the second data with the second sensor comprises obtaining inductance data with an induction sensor.

55. The method of claim 53, wherein:
obtaining the first data with the first sensor comprises obtaining first inductance data with a first induction sensor;
obtaining the second data with the second sensor comprises obtaining second inductance data with a second induction sensor; and
identifying the tool comprises identifying the tool based on a difference between the first and second inductance data.

56. The method of claim 53, wherein obtaining the first data with the first sensor comprises obtaining a torque data of an actuator controlling manipulation of an articulable body portion of the flexible elongate device with an encoder or force sensor.

57. The method of claim 56, wherein obtaining the second data with the second sensor comprises obtaining bending angle data of an articulable body portion of the flexible elongate device with a shape sensor; and further comprising:
estimating a stiffness of at least the tool based on the torque data and the bending angle data; and
wherein identifying the tool comprises identifying the tool based on the estimated stiffness.
